SEQUENCE LISTING (2 sequences)

SEQ ID NO: 1 - DNA, 29 bp, Homo sapiens
aacatactactcaaagacattcctgtctc

SEQ ID NO: 2 - DNA, 29 bp, Homo sapiens
aaaatgtctttgagtagtatgcctgtctc

(19) United States
(12) Patent Application Publication
(10) Pub. No.: US 2008/0279866 A1
(43) Pub. Date: Nov. 13, 2008

(54) Induction of immunosuppression by inhibition of ATM

(75) Inventors: John Iacomini, Somerville, MA (US); Jessamyn Bagley, Boston, MA (US)

Correspondence Address: LAW OFFICE OF MICHAEL A. SANZO, LLC, 15400 CALHOUN DR., SUITE 125, ROCKVILLE, MD, 20855, US

(73) Assignee: The Brigham and Women's Hospital, Inc., Boston, MA (US)

(21) Appl. No.: 11/889,940

(22) Filed: Aug. 17, 2007

Related U.S. Application Data
(60) Provisional application No. 60/840,037, filed on Aug. 25, 2006.

(51) Int. Cl. A61K 39/395; C12N 5/06; A61P 37/00; A61P 3/10; A61K 35/28; A61K 31/5377

(52) U.S. Cl. 424/173.1; 435/372.3; 424/577; 514/231.5

(57) ABSTRACT

The present invention is directed to methods of inducing immunosuppression in a patient by administering an inhibitor of the enzyme Ataxia telangiectasia mutated (Atm). The method may be used as a treatment for allergies, autoimmune diseases or lymphomas. It may also be used to prevent organ rejection in transplant patients and to treat or prevent graft versus host disease.

### CROSS REFERENCE TO RELATED APPLICATIONS

The present application claims priority to, and the benefit of, U.S. provisional application 60/840,037, filed on Aug. 25, 2006. This prior application is hereby incorporated by reference in its entirety.

### FIELD OF THE INVENTION

The present invention is directed to methods in which Atm inhibitors are used to treat or prevent a variety of diseases. The inhibitors will be especially useful with respect to autoimmune diseases, allergies, preventing organ rejection in transplant patients and in the treatment or prevention of graft versus host disease.

### BACKGROUND OF THE INVENTION

Ataxia telangiectasia (A-T) is a neurodegenerative disease that appears in childhood and is characterized by delayed development, poor balance, and slurred speech. About 20% of patients with A-T develop cancer, most frequently acute lymphocytic leukemia or lymphoma and many patients have a weakened immune system, making them susceptible to recurrent infections. There is no cure for A-T and most patients die in their teens or early 20s.

A-T is caused by mutations in the gene encoding Ataxia telangiectasia mutated (Atm), a member of the PI-3 kinase-like kinase family that includes ATR, DNA-PKcs and mTOR. It is well established that Atm plays a central role in cellular responses leading to repair of DNA double strand breaks (reviewed in Shiloh, Biochem. Soc. Trans. 29:661 (2001)). In addition, several observations suggest that Atm may also be involved in T cell function. A significant number A-T patients have immunodeficiencies that affect skin antigen test responses, responses to alloantigens or mitogens, and production of T cell-dependent IgE, IgA and IgG₄ antibodies (Lavin, et al., Annu. Rev Immunol 15:177 (1997); Schubert, et al., Clin. Exp. Immunol. 129:125 (2002); Nowak-Wegrzyn, et al. J. Pediatr. 144:505 (2004)). Defects in thymocyte development and a reduction in peripheral T cell numbers have also been observed in A-T patients (Datta, Indian J. Med. Res. 94:252 (1991); Giovannetti, et al., Blood 100:4082 (2002)) and Atm deficient mice (Atm−/− mice) (Barlow, et al., Cell 86:159 (1996); Borghesani, et al., Proc. Nat'l Acad. Sci. USA 97:3336 (2000)). These defects appear to be related to the role of Atm in T cells rather than the Atm deficient thymic environment in which these cells develop (Bagley, et al., Blood 104:572 (2004)). However, the mechanism by which Atm deficiency results in immunodeficiency is not known.

Defects in Atm may lead to abnormalities in cellular responses to reactive oxygen species (Rotman, et al., Bioessays 19:911 (1997); Ito, et al., Nature 431:997 (2004); Schubert, et al., Hum. Mol. Genet. 13:1793 (2004); Barlow, et al., Proc. Nat'l Acad. Sci. USA 96:9915 (1999)). Cells derived from A-T patients and Atm-deficient mice exhibit genomic instability and hypersensitivity to ionizing radiation and other treatments that generate ROS. ROS are also produced during normal metabolic activities such as T cell activation (Devadas, et al., J. Exp. Med. 195:59 (2002); Hildeman, et al., Immunity 10:735 (1999)). At nontoxic concentrations, ROS may play a role in signal transduction in T cells, but at higher levels they can inflict oxidative damage to cellular components, resulting cell death (Hildeman, et al., Immunity 10: 735 (1999); Hildeman, et al., J. Clin. Invest. 111:575 (2003)). A better understanding of the relationship between Atm deficiency, AT and ROS may lead to new therapeutic approaches to AT and other diseases in which activated T cells play a role.

### SUMMARY OF THE INVENTION

The present invention is based upon the discovery that the stimulation of normal T cells with anti-CD3 and anti-CD28 results in a significant proliferation of cells over a 72-hour period, whereas Atm deficient T cells fail to proliferate under the same conditions. Instead of inducing cellular proliferation, these agents induce apoptosis. The Atm deficient T cells were also found to have an impaired ability to respond to foreign alloantigens and normal T cells exposed to inhibitors of Atm (either the nonspecific inhibitor caffeine or the specific inhibitor KU-55933) were found to undergo apoptosis when exposed to anti-CD3 and anti-CD28. Overall, these results lead to the conclusion that ATM inhibitors may be used to block the biological actions of activated T cells and to thereby induce immunosuppression. This will be useful in the treatment of autoimmune diseases, allergies, in preventing the rejection of transplanted organs by host organisms and in preventing graft versus host disease in patients undergoing bone marrow transplantation. ATM inhibitors should also be useful in the treatment of immune cell related cancers, especially lymphocytic leukemia and T cell lymphomas.

Other results suggest that induction of apoptosis is due to an inability of Atm deficient cells to effectively cope with reactive oxygen species generated as the result of activating agents interacting with the T cell receptor. This is consistent with suggestions that agents scavenging reactive oxygen species should be useful in treating AT (see Background section).

In its first aspect, the present invention is directed to a method of inducing apoptosis in T cells by treating the cells with an effective amount of a drug that inhibits the enzyme Ataxia Telangiectasia mutated (Atm). The cells must also be contacted with an effective amount of an activating agent, i.e., an agent that binds to the T cell receptor thereby activating the cells, preferably after or concurrently to exposure to the Atm inhibitor. The term “effective amount” means a sufficient amount of Atm inhibitor and activating agent so that, within 72 hours, at least 20% of the T cells exposed to these drugs have undergone, or are undergoing, apoptosis as determined using standard assays for this process. If the method is being used by a scientist conducting studies in vitro, then the inhibitor and activating agent may be added to cell culture medium. If the method is being used in vivo, these drugs may be administered to a test subject or, alternatively, the Atm inhibitor may be administered and the activating agent may be supplied endogenously by the subject, i.e., a natural activator produced in vivo will be sufficient. Thus, in the latter case, the method would simply involve administering the Atm inhibitor. In a particularly preferred embodiment, organs undergoing transplantation will be exposed to the combination of an Atm inhibitor and an activating agent. This procedure is especially preferred in patients undergoing bone marrow transplant procedures as a method for reducing the number of alloreactive T cells and thereby reducing the likelihood of graft versus host disease. For example, the cells being transplanted may be exposed to an effective amount of Atm inhibitor for a period of from one to 72 hours and then subsequently exposed to a T cell activating agent for an additional one to 72 hours. Transplantation should occur within one week after exposure of the cells to the activating agent, and preferably within 72 hours after exposure.

Any agent that has been described in the art that both binds to the T cell receptor and causes activation may be used as the activating agent in the method described above including antibodies against CD3 and CD28. In addition, alloantigens or other antigens known to stimulate T cells may be employed.

Any type of agent that leads to a reduction in Atm enzymatic activity in T cells may be used as the Atm inhibitor in the method described above. This includes both agents that prevent the cellular synthesis of Atm, e.g., small inhibitory RNAs, as well as compounds that have been described in the art as inhibiting the activity of the enzyme directly. Although non-specific inhibitors such as caffeine and 2-aminopurine may be used, compounds that act more specifically on the Atm enzyme, such as those described in U.S. Pat. No. 7,049,313 and US 2005-0054657, are preferred. The most preferred compound is 2-morpholin-4-yl-6-thianthren-1-yl-pyran-4-one (KU-55933).

In another aspect, the invention is directed to a method of inducing an immunosuppressive state in a patient by administering a therapeutically effective amount of a drug that reduces the activity of Atm in the T cells of the patient. The same drugs described above in connection with methods of inducing apoptosis may be used in the method of inducing immunosuppression. A “therapeutically effective amount” is a sufficient quantity of drug to achieve a therapeutic objective. For example, in the treatment of an existing disease such as rheumatoid arthritis, sufficient drug should be given to alleviate at least one symptom associated with the disease, e.g., pain or inflammation. When dealing with an autoimmune disease, sufficient drug should be given to retard the progression of the disease or improve one or more of its symptoms. In the case of organ transplantation, enough drug should be given to reduce the likelihood of rejection due to an immune response triggered, in part, by the activation of T cells. A similar definition applies with respect to the treatment of graft versus host disease patients, where sufficient drugs should be given to block graft-generated cells from attacking host organs. Thus, in addition to depleting organs undergoing transplantation of alloreactive T cells, Atm inhibitors may be given to patients after transplantation to further reduce the likelihood of rejection.

The treatment method described above may be used in connection with any disease or condition where immunosuppression is desirable, including the treatment of patients for allergies, autoimmune diseases and cancers originating in immune cells, e.g., lymphomas. Specific autoimmune diseases that may be treated include asthma, multiple sclerosis, systemic lupus erythematosus, Hashimoto's thyroiditis, Grave's disease, inflammatory bowel disease, type 1 diabetes, psoriasis, scleroderma, and rheumatoid arthritis. As noted previously, the method may also be used to prevent acute or chronic organ transplant rejection or to treat or prevent graft versus host disease. The term “transplant patients” includes patients receiving a heart, lung, kidney, liver, pancreas or bone marrow (which for the purposes of the present invention is considered to be an organ). The drug may be administered systemically or, alternatively, may be implanted in a slow release formulation in close proximity to a transplanted organ. This should increase local concentration of the drug at the site where T cell activation is likely to occur. In the most preferred embodiment, the method is used in connection with patients undergoing bone marrow transplantation.

As discussed above, the invention includes improvements in a medical procedures in which a donor organ is transplanted into a host recipient, in which the organ is incubated in a solution containing an Atm inhibitor for a period of between one and 72 hours prior to transplantation and, preferably, also exposed to a T cell activating agent. The organ itself may be any of those described above and, as in all of the procedures described herein, the most preferred Atm inhibitor is KU-55933. The use of the procedure in BMT patients to reduce the likelihood of graft versus host disease is especially preferred.

In another aspect, the invention is directed to a therapeutic composition comprising both an Atm inhibitor in a first finished pharmaceutical container and a T cell activating agent that is also in a finished pharmaceutical container which may or may not be the same as the first finished pharmaceutical container. The preferred Atm inhibitor is KU-55933 and the preferred T cell activating agent is antibody against CD3, antibody against CD28 or an alloantigen. The package may include instructions for administering the drugs to a patient or to an organ for the treatment or prevention of any of the diseases or conditions described above. The instructions will include the dosage of inhibitor to be administered, along with additional information concerning treatment procedures. These instructions may appear as a package insert, on the finished pharmaceutical container or on the outside of other packaging.

### DETAILED DESCRIPTION OF THE INVENTION

The present invention is based upon the discovery that, following stimulation through the T cell receptor, Atm-deficient T cells and normal T cells in which Atm is inhibited, undergo apoptosis rather than proliferation. Apoptosis is prevented by scavenging reactive oxygen species (ROS) during activation. Atm therefore plays a critical role in T cell proliferation by regulating responses to ROS generated following T cell activation. The inability of Atm-deficient T cells to control responses to ROS is therefore the molecular basis of immunodeficiency associated with A-T and methods in which Atm is inhibited may be used to induce a similar state for the purpose of achieving therapeutic objectives.

A. Atm Inhibitors

Drugs inhibiting Atm may either be chemical compounds or small inhibitory RNA (siRNA) molecules. SiRNAs suitable for inhibiting Atm production have been described by Ouyang, et al. (Biochem. Biophys. Res. Commun. 337:875-880 (2005)), Nur-E-Kamal, et al. (J. Biol. Chem. 278:12475-12481 (2003)) and Casper et al., Cell 111:779-789 (2002)). Examples of sequences reported to be effective are: Antisense: AAC ATA CTA CTC AAA GAC ATT CCT GTC TC (SEQ ID NO:1) and Sense: AAA ATG TCT TTG AGT AGT ATG CCT GTC TC (SEQ ID NO:2).

Any pharmaceutically acceptable chemical compound that has been described in the art as inhibiting Atm may be used in connection with the present invention. Examples of relatively nonspecific inhibitors are caffeine and 2-aminopurine. However chemical compounds more specific in their action on Atm are preferred and have been described, for example, in U.S. Pat. No. 7,049,313 and in US 2005-0054657. Included among these are compounds of formula I:

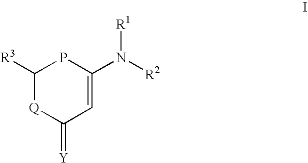

wherein: one of P and Q is O, and the other of P and Q is CH, where there is a double bond between whichever of Q and P is CH and the carbon atom bearing the R³ group; Y is either O or S; R¹ and R² together form, along with the nitrogen atom to which they are attached, a morpholino group; R³ is a first phenyl group, attached by a first bridge group selected from —S—, —S(═O)—, —S(═O)₂—, —O— and CR^{C1}R^{C2}—, to an optionally substituted second phenyl group; the first phenyl group and the second phenyl group being optionally further linked by a second bridge group selected from —S, —S(═O)—, —S(═O)₂, —O—, CR^{C1}R^{C2}—, —CR^{C1}R^{C2}CR^{C1}R^{C2}—, —C═O, —CR^{C1}R^{C2}S—, —CR^{C1}R^{C2}O—, —SCR^{C1}R^{C2}—, —OCR^{C1}R^{C2}—, —RC═CR—, or a single bond, which is bound adjacent the first bridge group on both groups so as to form an optionally substituted C₅₋₇ ring fused to both the first phenyl group and the second phenyl group, the first phenyl group being further optionally substituted;
R^{C1} and R^{C2} are independently selected from hydrogen, an optionally substituted C₁₋₇ alkyl group and an optionally substituted C₅₋₂₀ aryl group; wherein the first phenyl group in R³ optionally bears a substituent selected from the group consisting of an amino group, a hydroxy group, a halo group, an acylamido group, a sulfonamino group, an alkoxy group, an acylkoxy group, an alkyl group, a nitro group, a cyano group, a thiol group, an alkylthio group, and an acyl group; and wherein the second phenyl group in R³ optionally bears a substitutent selected from the group consisting of an acylamido group, an ester group, an amido group, an amino group, an acyl group, a sulfonamino group, an ether group, and a carboxy group.

The most preferred chemical compound is KU-55933 (2-morpholin-4-yl-6-thianthren-1-yl-pyran-4-one) that may be obtained as described in Hickson, et al. (Cancer Res. 64:9152-9159 (2004). This compound has been reported to be highly specific in its action on Atm and is presently under development as a cancer treatment by KuDOS Pharmaceuticals (Cambridge, England).

Methods for screening compounds for their ability to inhibit Atm have been described in the art (see e.g., U.S. Pat. No. 6,387,640) and may be used to identify additional compounds useful in any of the treatment methods described herein.

B. Making of Pharmaceutical Compositions

Atm inhibitors may be incorporated into pharmaceutical compositions in accordance with methods that are standard in the art (see e.g., Remingon's Pharmaceutical Sciences. Mack Publishing Co., (1990)). Formulations may be designed for delivery by any of the routes commonly used, with preparations designed for oral delivery being preferred. For oral compositions, e.g. tablets or capsules, the inhibitor should typically be present in an amount of between 0.01 and 100 mg. Although not preferred, other routes of administration may also be employed.

Atm inhibitors may be used in conjunction with any of the vehicles and excipients commonly employed in pharmaceutical preparations including water, salt solutions, alcohols, gum arabic, vegetable oils, benzo-alcohols, polyethylene glycol, gelatin, carbohydrates such as lactose, amylase, or starch; magnesium stearate; talc; salycic acid; paraffin; fatty acid esters; polymers; etc. The pharmaceutical preparations can be sterilized and, if desired, mixed with auxiliary agents such as: dispersants; lubricants; preservatives; stabilizers; wetting agents; emulsifiers; salts for influencing osmotic pressure; buffers; coloring agents; flavoring agents; and/or aromatic substances.

Solutions, particularly solutions for injection, can be prepared using water or physiologically compatible organic solvents such ethanol, 1,2-propylene glycol; polygycols; dimethylsulfoxides; fatty alcohols; triglycerides; partial esters of glycerine; and the like. The preparations can be made using conventional techniques that may include sterile isotonic saline, water, 1,3-butanediol, ethanol, 1,2-propylene glycol, polygycols mixed with water, ringers Ringer's solution etc.

C. Dosage Forms and Routes of Administration

The present invention is compatible with any route of administration including oral, peroral, internal, rectal nasal, lingual, transdermal, vaginal, intravenous, intraarterial, intramuscular, intraperitoneal, intracutaneus and subtaneous routes. Dosage forms that may be used include tablets, capsules, powders, aerosols, suppositories, skin patches, parenterals, sustained release preparations and oral liquids, including suspensions solutions and emulsions. The most preferred route of administration is oral. If desired, compositions, particularly compositions for injection, may be freeze-dried and lyophilizates reconstituted before administration. Dosage forms may include Atm inhibitors as the sole active ingredient or they may include other active agents as well. All dosage forms may be prepared using methods that are standard in the art and that are taught in reference works such as Remington's Pharmaceutical Sciences (Osol, A, ed. Mack Publishing Co. (1990)).

D. Treatment Methods

The methods described are directed to treating or preventing the development of one of the diseases or conditions described herein by suppressing the activity of a patient's T cells. In the case of treatments for an existing disease, successful treatment will be reflected in an improvement in one or more symptoms associated with the disease. For example, in the treatment of a rheumatoid arthritis, sufficient drug should be provided to reduce pain or swelling associated with this disease. When used to prevent organ rejection or graft versus host disease, the dose administered will be based upon the results of animal studies and clinical studies performed using methods well known in the art. In all cases, treatment methods and dosages will be selected by the attending physician based upon clinical considerations using methods that are well-known in the art.

E. Packaging of Therapeutic Compositions

As described previously, the pharmaceutical compositions containing Atm inhibitors and/or T cell activators may be placed in a finished pharmaceutical container and sold along with instructions to physicians regarding the use of the compositions in treating or preventing one of the diseases or conditions described herein. The compositions will be in a single package and, depending upon the intended route of delivery, may be in bottles, vials, ampoules, blister packs etc.

Instructions concerning the use of pharmaceutical compositions may be included on the container with the pharmaceutical composition or as a package insert. Alternatively, the instructions may be included on a box or other package in which the pharmaceutical composition is sold. In all cases, the instructions will indicate that the pharmaceutical compositions are to be administered for the purpose of preventing or treating one of the diseases or conditions described above. A description of the active ingredient(s) will also be included along with information concerning dosage and how the pharmaceutical composition should be administered.

### EXAMPLES

### Example 1

### ATM Inhibitors as Antagonists of Activated T Cells

The present example provides evidence suggesting that ATM inhibitors can be used to block the activity of activated T cells, including T cells activated as the result of being exposed to alloantigens. As such, the inhibitors are capable of suppressing the immune system and should be of use in treating diseases that may benefit from such suppression.

A. Methods

Mice: Heterozygous 129S6/SvEvTac-Atm^{tm1-Awb} mice (Atm−/−) were purchased from the Jackson Laboratory (Bar Harbor, Me.). An independently generated Atm knockout mouse model was used to confirm our observations (Borghesani, et al., Proc. Nat'l Acad. Sci. USA 97:3336 (2000)). All mice were housed under microisolator conditions in autoclaved cages and were maintained on irradiated feed and autoclaved acidified drinking water. All sentinel mice housed in the same colony were free of viral antibodies. Four- to 6-week-old mice were used in all experiments.

Purification and Stimulation of T Cells: Splenocytes were harvested from 4-6 week old Atm−/− mice or wild-type littermates bred in our animal facility. In some experiments C57BL/6 mice were used as a source of wild-type T cells. Red blood cells were lysed using ACK lysing buffer (Cambrex, Walkerville, Md.) for 3 minutes at room temperature. In experiments in which T cells were purified, splenocytes were incubated with anti-CD4 (GK1.5, Dialynas, et al., J. Immunol. 131:2445 (1983)) and anti-CD8 (2.43, Sarmiento, et al., J. Immunol. 125:2665 (1980)) antibodies for 30 min at 4° C. Cells were then washed and incubated with magnetic beads conjugated to anti-rat IgG antibody, prior to positive selection by MACS according to the manufacturer's instructions (Miltenyi Biotech, USA Auburn Calif.).

Cells were then labeled for 20 minutes with 2 μM 5(6)-Carboxyfluorescein diacetate N-succinimidyl ester (CFSE, Sigma-Aldrich) in Hank's balanced salt solution (HBSS, Mediatech, Herndon, Va.). Cells were plated at a concentration of 2-5×10⁶/ml in Dulbecco's modified eagle's medium (DMEM (Mediatech) supplemented with 15% heat-inactivated fetal calf serum (Sigma-Aldrich, St. Louis, Mo.), penicillin (100 U/ml), streptomycin (100 μg/ml), 2 mM L-glutamine, 10 mM Hepes, 0.1 mM nonessential amino acids, 1 mM sodium pyruvate (Mediatech) (complete DMEM). Cells were treated with 1 μg/ml anti-CD3 antibody (2C11 (30)) and 1 μg/ml anti-CD28 antibody (37.51, Gross, et al., J. Immunol. 149:380 (1992)). Cells were cultured at 37 C, 5% CO₂ for the indicated time. For studies of Atm−/− T cell proliferation in response to mitogens, purified T cells were cultured with 2 μg/ml Concanavalin A or 10 ng/ml PMA and 1 μg/m ionomycin.

Stimulation of T cells in the Presence of Caffeine: Splenocytes were harvested from C57BL/6 mice (Jackson Laboratories) and re-suspended at 3×10⁶ cells/ml in complete DMEM with 1 μg/ml anti-CD3 and CD28 and various concentrations of caffeine (Acros Organics, N.J.), or the same volume of water. Some cultures were, in addition, treated with 2 mM N-acetyl cysteine (Sigma-Aldrich).

Stimulation of T cells in the Presence of KU-55933: T cells were purified from C57BL/6 mice (Jackson Laboratories) and re-suspended at 1.5×10⁶ cells/ml in complete DMEM with 1 μg/10⁶ cells anti-CD3 and CD28 and the indicated concentration of KU-55933 (KuDos Pharmaceuticals) or the same volume of DMSO (Sigma-Aldrich). Some cultures were in addition treated with 4 mM N-acetyl cysteine (Sigma-Aldrich).

Flow Cytometry: Cells were harvested, washed in HBSS and stained with Annexin-V phycoerythrin (BD Biosciences), or Annexin V-biotin (R&D Systems) in addition to streptavidin conjugated phycoerythrin according to the manufacturer's instructions. Cells were then stained with anti-CD4-APC (RM4-5, BD Biosciences) or anti-CD8-APC (53-6.7 BD Biosciences) and 7-actinomycin D (Sigma-Aldrich) or propidium iodide (Sigma-Aldrich). All analysis was performed using. FloJo software (TreeStar Inc).

Western Blots: Purified T cells from C57BL/6 mice were stimulated as described. After 13 hours, cells were counted and washed with phosphate buffered saline. Equal numbers of cells were lysed with Cytobuster Reagent (Novagen, Madison, Wis.) in the presence of Protease Inhibitor Cocktail (Roche Indianapolis, Ind.) for each sample. Lysates were separated by 3-8% Tris-Acetate gels under denaturing conditions, and transferred to nitrocellulose membranes. Membranes were blocked with 5% non-fat milk for 1 hour and incubated with 1:1000 dilution of anti-ATM (MAT3) or 1:500 dilution of anti-pS1981-ATM overnight at 4° C. The membrane was subsequently incubated with Horseradish peroxidase conjugated secondary antibody, and developed with ECL reagent (GE Healthcare Bio-Sciences Corp, Piscataway, N.J.).

In Vivo Proliferation: T cells were purified from Atm−/− mice or Atm+/+controls and labeled with CFSE as described. 10⁶ labeled T cells were injected into either BALB/c or C57BL/6 recipients. 72 hours later recipients were sacrificed and splenocytes were examined for CFSE fluorescence by flow cytometry.

B. Results

To examine the role of Atm in T cell function, we analyzed responses of Atm deficient T cells following stimulation through the T cell receptor (TCR). T cells were purified from the spleens of either Atm−/− or normal littermate mice, labeled with carboxyfluorescein diacetate, succinimidyl ester (CFSE), and stimulated in vitro with antibodies specific for CD3 and CD28. Following stimulation, T cells were harvested, stained with annexin-V and 7-aminoactinomycin D (7-AAD), and then analyzed by flow cytometry. CFSE intensity, which is reduced by one-half with each cell division, was used to examine proliferation over time by flow cytometry after gating out annexin-V⁺ apoptotic cells and 7-AAD⁺ dead cells.

Stimulation of T cells from ATM+/+normal littermates with anti-CD3 and anti-CD28 resulted in significant proliferation over a 72-hour period. In contrast, Atm deficient T cells failed to proliferate under the same conditions. Analysis of 7AAD T cells revealed that stimulation of Atm−/− T cells resulted in apoptosis rather than proliferation. T cells from normal littermate mice proliferated following stimulation with anti-CD3 and anti-CD28, as expected, and 72 hours after stimulation relatively few T cells stained with annexin-V. In contrast, following stimulation, the majority of T cells from Atm deficient mice failed to proliferate and became annexin-V⁺. After 12 hours of stimulation, similar numbers of Atm−/− and Atm+/+were annexin-V⁺, indicating that Atm−/− T cells did not intrinsically express higher phosphatidylserine levels on the cell membrane. Both Atm deficient CD4 T cells and Atm deficient CD8 T cells were susceptible to apoptosis induction following stimulation with anti-CD3 and CD28 when compared to, normal littermate CD4 and CD8 T cells.

Stimulation of unfractionated splenocytes from Atm−/− mice with anti-CD3 and CD28 similarly resulted in T cell apoptosis rather than proliferation, indicating that the results observed were not related to the effects of T cell purification. Stimulation of Atm−/− T cells with anti-CD3 alone also resulted in greater levels of apoptosis than observed in Atm+/+ T cells. Therefore, the observed defect in the ability to proliferate was not related to a defect in CD28 signaling. Similar results were also observed using. T cells from a second strain of Atm deficient mice (Borghesani, et al., Proc. Nat'l Acad. Sci. USA 97:3336 (2000)) and were therefore not specific to a particular strain of Atm mutants.

To examine whether signaling downstream of the TCR resulted in apoptosis of Atm deficient T cells, T cells from Atm−/− mice and wild-type controls were stimulated with phorbol 12-myristate 13-acetate (PMA) and ionomycin. PMA and ionomycin mimic signaling through the TCR by activating protein kinase C and increasing cytoplasmic free calcium levels. Seventy-two hours after stimulation of Atm deficient T cells with PMA and ionomycin few annexin-V⁻ AAD⁻ Atm deficient T cells were present in the cultures. Stimulation of Atm deficient T cells with PMA/ionomycin induced apoptosis based on staining with annexin-V as was observed following stimulation with anti-CD3 and CD28 stimulation. Therefore, signals downstream of the TCR result in apoptosis in the absence of Atm.

We next asked whether apoptosis in Atm deficient T cells following stimulation was a result of a general defect in the ability of Atm−/− T cells to proliferate. Stimulation of Atm deficient T cells with the mitogen concanavalin-A (Con A) resulted in proliferation. Seventy-two hours after stimulation with Con A, the number of annexin-V⁻ T cells was similar in cultures containing either Atm deficient or normal littermate T cells. Furthermore, the number of cell divisions following stimulation with Con A was similar for Atm deficient and normal littermate T cells. These data suggest that induction of apoptosis following stimulation of Atm deficient T cells is specifically related to signaling through the TCR and is not the result of a defect in the ability of Atm deficient T cells to proliferate.

Atm deficient mice have been reported to exhibit defects in T cell development (Barlow, et al., Proc. Nat'l Acad. Sci. USA 96:9915 (1999)). We therefore set out to examine whether Atm plays a role in the activation of T cells from normal mice following stimulation through the TCR. Upon activation, Atm becomes phosphorylated at serine residue 1981 (Bakkenist, et al., Nature 421:499 (2003)). Western blot analysis of lysates from T cells purified from C57BL/6 mice indicated, that only low levels of phosphorylated Atm could be detected in un-stimulated T cells. In contrast, stimulation with either anti-CD3 and anti-CD28, PMA/ionomycin or Con A resulted in an increase in the amount of phosphorylated Atm, indicating that Atm is activated in wild-type T cells following stimulation through the TCR.

We next examined whether in vivo responses to alloantigen were impaired in the absence of Atm by analyzing the ability of ATM deficient and wild type T cells to proliferate in response to alloantigens. T cells were purified from Atm+/+ or Atm−/− mice, CFSE labeled and then adoptively transferred into allogeneic BALB/c or syngeneic C57BL/6 recipients. 72 hours after transfer the spleens of recipients were examined for the presence of CFSE labeled cells by flow cytometry. A fraction of Atm+/+ T cells proliferated when adoptively transferred into allogeneic BALB/c mice. In contrast, we were unable to detect viable Atm−/− T cells that had proliferated when adoptively transferred into BALB/c hosts. This was not a result of an impaired ability of Atm−/− T cells to survive adoptive transfer, as there was no significant difference in the number of non-dividing. CFSE labeled T cells when Atm−/− and Atm+/+populations were compared (P=0.17). These data suggest that ATM deficient T cells also exhibit defects in proliferation following signaling through the TCR in vivo.

We reasoned that defects in Atm−/− T cells might reflect a requirement of mature T cells for Atm. To test this we examined the effect of inhibition of Atm on wild-type T cells. We therefore examined the ability of T cells from C57BL/6 mice to proliferate in response to anti-CD3 and anti-CD28 antibodies in the presence of caffeine, which is known to inhibit Atm. Caffeine concentrations of 1 mM inhibit ATM but not ATR function in vitro and caffeine concentrations of 3 mM inhibit both ATM and ATR function by 50% (Sarkaria, et al Cancer Res. 59:4375 (1999); Kaufmann, et al., Mutat. Res. 532:85 (2003)). Stimulation of C57BL/6 T cells with anti-CD3 and CD28 in the presence of caffeine at concentrations as low as 1 mM induced T cell apoptosis. Increasing, the concentration of caffeine led to a dose dependent increase in the frequency of T cells undergoing apoptosis. Analysis of T cell proliferation over-time revealed that the addition of 2.5 mM caffeine to T cells stimulated with anti-CD3 and CD28 had no effect on cell viability at 24 hours. At 48 and 72 hours after stimulation, only T cells which did not divide remained annexin-V negative.

Since caffeine can also affect other members of the PI-3 kinase-like kinase family, we next examined the effect of a specific Atm inhibitor, KU-55933 (2-morpholin-4-yl-6-thianthren-1-yl-pyran-4-one; Hickson, et al Cancer Res. 64:9152 (2004)) on activation of T cells following stimulation through the TCR. Cellular activity of KU-55933 has been demonstrated through both radiosensitization experiments and the abrogation of ionizing-radiation-dependent phosphorylation of known ATM targets including, p53, H2AX and NBS1. This compound is highly specific for Atm, and does not inhibit of other PI-3 kinase-like kinase family members. Stimulation of C57BL/6 T cells with anti-CD3 and CD28 in the presence of KU-55933 at concentrations as low as 10 μM induced T cell apoptosis. Increasing the concentration of KU-55933 led to a dose dependent increase in the frequency of T cells undergoing apoptosis. Analysis of T cell proliferation over-time revealed that the addition of 20 μM KU-55933 to T cells stimulated with anti-CD3 and CD28 had no effect on cell viability at 24 hours. However, 48 and 72 hours after stimulation, KU-55933 treated T cells underwent significantly less proliferation than untreated controls. These data indicate that the inhibition of Atm in wild-type T cells induces apoptosis following stimulation through the TCR. Therefore, Atm is required for T cell proliferation following signaling through the TCR.

In addition to its role in regulating cell responses to gamma irradiation, Atm has been suggested to be involved in regulation of responses to oxidative stress by activating pathways of reactive oxygen species (ROS) metabolism (Ito, et al Nature 431:997 (2004); Barlow, et al., Proc. Nat'l Acad. Sci. USA 96:9915 (1999); Hammond, et al., J. Biol. Chem. 278:12207 (2003)). ROS cause single strand DNA breaks, and have been shown to activate Atm. It has also been suggested that T cell activation leads to the generation of ROS. We hypothesized that induction of apoptosis in Atm deficient T cells following stimulation through the TCR may be related to the inability of Atm deficient T cells to regulate responses to ROS generated following activation. To test this hypothesis, Atm deficient T cells were stimulated with anti-CD3 and CD28 in either the presence or absence of the anti-oxidative agent N-acetyl cysteine (NAC). The addition of 2 mM NAC to Atm deficient T cells stimulated with anti-CD3 and CD28 restored the ability of Atm deficient T cells to undergo proliferation. In the presence of NAC, proliferation of Atm deficient T cells was similar to that observed for T cells from normal littermate controls.

We next examined whether the induction of apoptosis in normal T cells following stimulation with anti-CD3 and CD28 in the presence of caffeine was prevented by the anti-oxidative agent NAC. Apoptosis induced in normal T cells following stimulation with anti-CD3 and CD28 in the presence of 2.5 mM caffeine was also prevented by the addition of 2 mM NAC. Proliferation of normal T cells stimulated with anti-CD3 and CD28 in the presence of caffeine and NAC was similar to proliferation in the presence of anti-CD3 and CD28 alone. Similarly, we examined whether induction of apoptosis following T cell stimulation in the presence of KU-55933 could be overcome by the addition of NAC. Apoptosis induced in normal T cells following stimulation with anti-CD3 and CD28 in the presence of 20 μM KU-55933 was prevented by the addition of NAC. These data demonstrate that induction of apoptosis in normal T cells following stimulation through the TCR in the presence Atm inhibitors is due to the generation of ROS. In addition, these data suggest that Atm plays a critical role in regulating responses to ROS generated during stimulation of T cells through the TCR.

C. Discussion

Consistent with the hypothesis that immunodeficiencies in Atm−/− T cells are cell intrinsic (Bagley, et al., Blood 104:572 (2004)), we observed that mature T cells derived from Atm deficient animals underwent apoptosis rather than proliferation in response to TCR stimulation. This was not due to a general defect in the ability of Atm−/− T cells to proliferate, since Atm−/− T cells proliferated normally following stimulation with the mitogen ConA. Con A has been shown to signal through the TCR, and to activate additional cell survival pathways through Akt/PKB (Pongracz, et al., Mol. Immunol. 39:1013 (2003)). This suggests that while stimulation through the TCR leads to apoptosis in Atm−/− cells, the simultaneous activation of cell survival pathways may be sufficient to prevent T cell death following stimulation.

Although the death of Atm−/− T cells in response to stimulation through the TCR was not due to a general inability of these cells to proliferate, Atm−/− mice display aberrant T cell differentiation (Barlow, et al., Cell 86:159 (1996)). To eliminate the possibility that developmental defects in Atm−/− T cells alters their proliferation in response to TCR stimulation, we next examined the role of Atm in wild-type T cells. We first used the classic inhibitor of Atm, caffeine, in cultures of wild-type T cells stimulated with antibodies specific for CD3 and CD28. As observed in Atm−/− T cells, wild-type T cells in which Atm was inhibited by caffeine underwent apoptosis rather than proliferation following stimulation through the TCR. While caffeine has been widely used to inhibit Atm (Sarkaria, et al., Cancer Res 59:4375 (1999)), it also inhibits other members of the PI-3-kinase like kinase family including. ATR and DNA-PK (Block, et al., Nucleic Acids Res. 32:1967 (2004)). We therefore next used the compound KU-55933, which specifically inhibits Atm, but not other members of the PI3K like-kinases (Hickson, et al., Cancer Res. 64:9152 (2004)) in culture with wild-type T cells. Our results demonstrate that specific inhibition of Atm in wild-type T cells results in the inability of mature T cells to proliferate following stimulation through the TCR. Since wild-type T cells mature in the presence of Atm, they have no intrinsic defect in the ability to proliferate after stimulation through the TCR. Thus, our data demonstrate that Atm is essential for the function of mature wild-type T cells.

We hypothesized that Atm was required in activated T cells to control cellular responses to ROS generation. When the reactive oxygen species scavenger NAC was added to cultures containing both stimulated Atm−/− T cells, and wild-type cells in which Atm was inhibited resulted in normal proliferation, and prevention of cell death. The inability of T cells in which Atm is absent or inhibited to proliferate in response to TCR stimulation is specific to the role of Atm in the control of ROS.

Our data support a model in which stimulation of T cells through the TCR results in ROS production, the cellular response to which is controlled by Atm. In the absence of Atm, ROS production leads to the induction of apoptosis. These data strongly suggest that Atm plays a critical role in T cell activation by regulating the cellular response to ROS following stimulation through the TCR. We have shown that in wild-type T cells, inhibition of Atm promotes apoptosis and prevents proliferation in an ROS dependent manner. These data place Atm in a central role in T cell responses to the generation of ROS.

Our data may also provide insight into the molecular basis of immunodeficiency associated with A-T. Based on our data in Atm deficient mice, we suggest that immunodeficiency associated with A-T is caused by the inability of Atm-deficient T cells to control responses to ROS generated following stimulation through the TCR. This defect results in induction of apoptosis rather than proliferation of T cells. Proliferation is required in order for activated T cells to gain effector function. The observation that scavenging reactive oxygen species restores T cell proliferation in Atm deficient T cells suggests clinically relevant therapies for the immunodeficiency associated with A-T. Because of the critical role of Atm in T cell activation following stimulation through the TCR, our results also suggest that pathways regulated by Atm may form the basis for the development of novel immunosuppressive drugs.

### Example II

### Inhibition of ATM for Prevention of GVHD and Tolerance Induction

Allogeneic bone marrow transplantation (BMT), is used clinically for a wide range of disorders including malignancy and repair of congenital genetic abnormalities. One of the major complications of BMT is the development of graft vs. host disease (GVHD) in which the T cells from the donor bone marrow inoculums respond to and destroy host tissue. The likelihood of developing. GVHD rises with age, with an incidence of 20% in the pediatric population and rising to 70% of BMT patients older than 50. The severity of GVHD can vary, and is classified from stage I to stage IV by symptoms. The most important factor correlating, with severity of GVHD is the degree of HLA disparity. With HLA-identical siblings used as bone marrow donors, incidence of moderate-to-severe acute GVHD ranges from less than 10% to 60%, depending on prophylaxis and other risk factors. Incidence of grades II-IV acute GVHD increases to 70-75% with one HLA antigen mismatch and up to 90% with 2-3 HLA antigen mismatch. GVHD causes both substantial morbidity in less severe cases, and substantial mortality among, BMT recipients. The survival rate is 90% in grade 0-I, 60% in grade II-III, and 0 in grade IV. Fatality mainly results from infections, hemorrhages, and hepatic failure. Thus, GVHD remains one of the major complications of BMT, and substantially limits the clinical use of this life-saving therapy.

Currently, the consequences of GVHD are treated with generalized immunosuppressives such as methotrexate and cyclosporine. Even when effective, these treatments expose patients to additional risks of infection, organ damage and malignancy associated with the long-term use of immunosuppressive agents. Thus, control of GVHD does not always lead to increased survival as a result of an increase in fatal infection. Currently the best approach to GVHD is prophylactic treatment of BMT patients.

Calcineurin inhibitors have been used in GVHD prophylaxis, but in addition to not being completely effective, these inhibitors have a toxicity to organs which limits their utility. Methotrexate and mycophenolate are also used to prevent the proliferation of alloreactive T cells while agents such as alemtuzumab and anti-thymocyte globulin are used to decrease the number of donor T cells. GVHD can best be prevented by the depletion of donor T cells from the bone marrow graft prior to treatment. However, while this significantly reduces the incidence of GVHD, it also appears to reduce the efficiency of bone marrow engraftment and leads to an increased risk of bone marrow failure and subsequent mortality. Therefore, we hypothesize that the development of treatments that specifically deplete the T cells capable of mediating GVHD, while leaving the remainder of donor T cells (which may participate in bone marrow engraftment) intact, may lead to effective new therapies for the prevention of GVHD.

It has been previously shown that ATM is critical for T cell survival following stimulation at the antigen specific T cell receptor (TCR). When T cells are stimulated in the presence of an agent the specifically inhibits ATM, they fail to proliferate and, instead, undergo apoptosis. This response appears to berated to the generation of oxidative stress following stimulation through the TCR.

In recent unpublished preliminary data, we have found that while alloreactive T cells normally produce IL-2, IFN-g and IL-4 in response to stimulation with alloantigen, they fail to produce these cytokines when treated with an ATM inhibiting agent in addition to alloantigen. As a result, we hypothesized that treatment of T cells with a combination of alloantigen to stimulate cells through the TCR, and an ATM inhibiting agent (KU55933) which induces apoptosis following TCR stimulation, would lead to the selective deletion of alloreactive T cells. In addition, we reasoned that T cells treated in this way would be unable to mediate GVHD.

B. Experimental Protocol and Results

To test this, Balb/c mice were lethally irradiated one day prior to BMT with C57BL/6 bone marrow. 48 Hours after BMT, mice received splenocytes treated with allogeneic stimulators, allogeneic stimulators and KU55933, or no stimulators and KU55933. Three out of four animals that received T cells stimulated with alloantigen rapidly developed severe GVHD, and died within 27 days of BMT. In contrast, three out of four animals that received T cells stimulated with alloantigen in addition to an agent that inhibits ATM survived long-term.

This suggests that inhibition of ATM combined with stimulation through the TCR results in inactivation of alloreactive T cells. To confirm that this effect was specific to stimulated T cells, we also treated unstimulated splenocytes with KU55933, the ATM inhibiting agent. In these mice we observed the development of GVHD at 30-40 days, suggesting that in the absence of stimulation, ATM inhibition did not affect the alloreactive T cells. Thus, these data suggest that the combination of stimulation through the TCR and ATM inhibition can specifically deplete alloreactive T cells and prevent GVHD while leaving un-stimulated T cells unaffected.

C. Discussion

Induction of tolerance to organ allografts is one of the major goals of transplantation research. It has long been known that the induction of mixed hematopoietic chimerism through allogeneic BMT results in the induction of tolerance to organ transplants. However, because of the risk of GVHD, and engraftment failure, this technique is currently to risky to be used to induce tolerance. It is possible therefore that the prevention of GVHD through ATM inhibition might allow this technique to be used in the clinic.

In addition, we have recently determined that mature T cells expressing alloantigen are capable of inducing tolerance without the need for any other alloantigen expressing cells. This observation suggests that if GVHD induced by the introduction of alloreactive mature T cells could be effectively prevented, the introduction of mature T cells from a donor could induce tolerance to solid organ grafts in treated recipients. We therefore suggest that treating alloantigen expressing mature T cells with ATM inhibitors which specifically deplete alloreactive T cells prior to introduction into recipients may lead to the induction of tolerance without GVHD.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

What is claimed is:

1. A method inducing the apoptosis of T cells comprising:
a) treating said T cells with an effective amount of a drug that inhibits the enzyme Ataxia telangiectasia mutated (Atm) in said cells; and
b) exposing said T cells to an effective amount of an agent that binds to the T cell receptor and thereby activates said T cells.

2. The method of claim 1, wherein said agent that reduces the activity of Atm is KU-55933.

3. The method of claim 1, wherein said agent that binds to said T cell receptor is antibody against CD3, antibody against CD28 or an alloantigen.

4. The method of claim 1, wherein said T cells are present in bone marrow.

5. The method of claim 4, further comprising transplanting said bone marrow into a patient.

6. The method of claim 5, wherein said agent that reduces the activity of Atm is KU-55933 and said agent that binds to said T cell receptor is antibody against CD3, antibody against CD28 or an alloantigen.

7. A method of inducing an immunosuppressive state in a patient, comprising administering to said patient a therapeutically effective amount of a drug that reduces the activity of Atm in the T cells of said patient.

8. The method of claim 7, wherein said drug is administered to said patient as a treatment for either an autoimmune disease, an allergy or a lymphoma.

9. The method of claim 8, wherein said disease or condition is selected from the group consisting of: asthma; multiple sclerosis; systemic lupus erythematosus; Hashimoto's thyroiditis; Grave's disease; inflammatory bowel disease; type 1 diabetes; psoriasis; scleroderma; and rheumatoid arthritis.

10. The method of claim 7, wherein said drug is administered to an organ transplant patient to prevent acute or chronic transplant rejection or to treat or prevent graft versus host disease.

11. The method of claim 10, wherein said drug is administered to a patient that has undergone transplant of the heart; lung; kidney; liver; pancreas; or bone marrow.

12. The method of claim 11, wherein said drug is KU-55933.

13. In a medical procedure in which a donor organ is transplanted into a host recipient, the improvement comprising exposing said organ to a drug that reduces the activity of Atm in T cells for a period of 1.0-72 hours prior to transplantation.

14. The improvement of claim 13, further comprising exposing said organ to an agent that activates T cells for a period of 1.0-72 hours prior to transplantation.

15. The improvement of claim 14, wherein said drug that reduces the activity of Atm in T cells is KU-55933 and said agent that activates T cells is antibody against CD3, antibody against CD28 or an alloantigen.

16. The improvement of claim 15, wherein said organ is selected from the group consisting of: heart; lung; kidney; liver; pancreas; and bone marrow.

17. The method of claim 16, wherein said drug is implanted in a slow release formulation in close proximity to said organ after transplantation.

18. A therapeutic package comprising:
a) a drug that reduces the activity of Atm in a first finished pharmaceutical container; and
b) an agent that activates T cells in a finished pharmaceutical container that may or may not be the same as said first finished pharmaceutical container.

19. The therapeutic package of claim 18, wherein said drug that reduces the activity of Atm is KU-55933.

20. The therapeutic package of claim 19, wherein said agent that activates T cells is antibody against CD3, antibody against CD28 or an alloantigen.
